SEQUENCE LISTING (24 sequences)

SEQ ID NO: 1 - DNA, 323 bp, Artificial Sequence
  Synthetic Sequence
ctaccgccttccttgtacaatcatctgatgatgtatcatctgactgcttttccactttcaccgtttacaaatagtttccaaataaaccccaacaaaaaagagaaaggaaaaaataaataaaaaaaaagataaagatatcgttgggaatgaaattctgtcatttgtatcccctttgacagaaaacggagggatctattgatgtattttaaaaattttatccgtcgggactgacggggctcgaacccgcagcttccgccttgacagggcggtgctctgaccaattgaactacaatcccagggaaataaagaaaagtgtacaacag

SEQ ID NO: 2 - DNA, 304 bp, Artificial Sequence
  Synthetic Sequence
gaaccctctttactgttcaaagagaaaaaaaagtcttttttttatttaattttaataagatcttgccttagtgtagtcacatattagacacttaccccctgttttatttgaatttcatttatgaaatgctttattgactcatttcatatcatggatcaaagaagttaaattcaaaatcggcagggtatacccttttttcgaaacgaaatacgaagaaaagttaccatagaactctttggattatatattcttttccaccctttcttttcgataatctaccgccttccttgtacaatcatctgat

SEQ ID NO: 3 - DNA, 26 bp, Artificial Sequence
  Synthetic Sequence
catctgatgatgtatcatctgactgc

SEQ ID NO: 4 - DNA, 24 bp, Artificial Sequence
  Synthetic Sequence
gaaccctctttactgttcaaagag

SEQ ID NO: 5 - DNA, 24 bp, Artificial Sequence
  Synthetic Sequence
ggattgtagttcaattggtcagag

SEQ ID NO: 6 - DNA, 24 bp, Artificial Sequence
  Synthetic Sequence
gtagttcaattggtcagagcaccg

SEQ ID NO: 7 - DNA, 22 bp, Artificial Sequence
  Synthetic Sequence
tcaattggtcagagcaccgccc

SEQ ID NO: 8 - DNA, 24 bp, Artificial Sequence
  Synthetic Sequence
tggtcagagcaccgccctgtcaag

SEQ ID NO: 9 - DNA, 24 bp, Artificial Sequence
  Synthetic Sequence
accgccctgtcaaggcggaagctg

SEQ ID NO: 10 - DNA, 24 bp, Artificial Sequence
  Synthetic Sequence
cctgtcaaggcggaagctgcgggt

SEQ ID NO: 11 - DNA, 24 bp, Artificial Sequence
  Synthetic Sequence
caaggcggaagctgcgggttcgag

SEQ ID NO: 12 - DNA, 23 bp, Artificial Sequence
  Synthetic Sequence
cggaagctgcgggttcgagcccc

SEQ ID NO: 13 - DNA, 21 bp, Artificial Sequence
  Synthetic Sequence
cccgtcagtcccgacggataa

SEQ ID NO: 14 - DNA, 23 bp, Artificial Sequence
  Synthetic Sequence
tcaatagatccctccgttttctg

SEQ ID NO: 15 - DNA, 19 bp, Artificial Sequence
  Synthetic Sequence
aacagggggtaagtgtcta

SEQ ID NO: 16 - DNA, 23 bp, Artificial Sequence
  Synthetic Sequence
aagggtataccctgccgattttg

SEQ ID NO: 17 - DNA, 24 bp, Artificial Sequence
  Synthetic Sequence
gagttctatggtaacttttcttcg

SEQ ID NO: 18 - DNA, 26 bp, Artificial Sequence
  Synthetic Sequence
ggtagattatcgaaaagaaagggtgg

SEQ ID NO: 19 - DNA, 25 bp, Artificial Sequence
  Synthetic Sequence
ggcggtagattatcgaaaagaaagg

SEQ ID NO: 20 - DNA, 23 bp, Artificial Sequence
  Synthetic Sequence
aggaaggcggtagattatcgaaa

SEQ ID NO: 21 - DNA, 25 bp, Artificial Sequence
  Synthetic Sequence
gtacaaggaaggcggtagattatcg

SEQ ID NO: 22 - DNA, 23 bp, Artificial Sequence
  Synthetic Sequence
tgtacaaggaaggcggtagatta

SEQ ID NO: 23 - DNA, 25 bp, Artificial Sequence
  Synthetic Sequence
tgattgtacaaggaaggcggtagat

SEQ ID NO: 24 - DNA, 22 bp, Artificial Sequence
  Synthetic Sequence
gatgattgtacaaggaaggcgg

(19) United States
(12) Patent Application Publication
(10) Pub. No.: US 2015/0329856 A1
(43) Pub. Date: Nov. 19, 2015

(54) METHODS AND SYSTEMS FOR THE GENERATION OF A PLURALITY OF SECURITY MARKERS AND THE DETECTION THEROF

(71) Applicant: APDN (B.V.I.) Inc., Tortola (VG)

(72) Inventors: Minghwa Benjamin LIANG, Stony Brook, NY (US); James A. HAYWARD, Stony Brook, NY (US); Lawrence JUNG, Dix Hills, NY (US)

(73) Assignee: APPLIED DNA SCIENCES, INC., Stony Brook, NY (US)

(21) Appl. No.: 14/695,228

(22) Filed: Apr. 24, 2015

Related U.S. Application Data
(63) Continuation of application No. 12/690,799, filed on Jan. 20, 2010, now abandoned.
Application No. 12/690,799 is a continuation-in-part of application No. 12/465,450, filed on May 13, 2009.
Application No. 12/465,450 is a continuation-in-part of application No. 11/954,030, filed on Dec. 11, 2007, now Pat. No. 8,372,648.
Application No. 11/954,030 is a continuation-in-part of application No. 11/437,265, filed on May 19, 2006, now abandoned.
Application No. 11/437,265 is a continuation-in-part of application No. 10/825,968, filed on Apr. 15, 2004, now abandoned.
(60) Provisional application No. 60/877,875, filed on Dec. 29, 2006.
(60) Provisional application No. 60/877,869, filed on Dec. 29, 2006.
(60) Provisional application No. 60/874,425, filed on Dec. 12, 2006.
(60) Provisional application No. 60/682,976, filed on May 20, 2005.
(60) Provisional application No. 60/463,215, filed on Apr. 16, 2003.

(51) Int. Cl. C12N 15/10

(52) U.S. Cl. CPC C12N 15/1096

(57) ABSTRACT

This invention pertains to methods for generating large quantities of DNA security markers by combinatorial variation techniques using polymorphic fragment length DNA for unique identification security marker applications such as explosive ink used in dye/smoke pack and cash carrying boxes.

### CROSS REFERENCE

This application is a ______ each of the patent applications being hereby incorporated by reference.

### FIELD

This invention pertains to methods and systems for generating a large number of security and/or authentication markers using a single template and the detection thereof.

### BACKGROUND

In addition to its role of being the blueprint for biological organisms, DNA has also been widely used as security markers in many applications such as disclosed in U.S. Pat. No. 6,312,911, U.S. Pat. No. 6,030,657, U.S. Pat. No. 5,643,728, and, GB 2390055. In U.S. Pat. No. 6,312,911, Bancroft et al. use DNA fragments to encrypt secret messages, where every three DNA bases represented either a letter or a symbol. The encoded message was then decoded by sequencing the DNA fragment in the security marker followed by referring to an encryption reference table for decoding. The secrecy of the encryption totally relies on the analysis using flanking primers and an encryption translation table after sequencing. Although this technique is familiar to those skilled in the art of molecular biology, it is not meant for generating large number of individualized security markers.

In Butland et al, U.S. Pat. No. 6,030,657, the labeling/marking technique utilized encapsulated biomarkers, such as encapsulated DNA, further labeled with infrared (IR) markers to label products for countering product diversion and product counterfeiting. In this patent, the DNA biomarker was a secondary consideration for security and DNA sequencing was needed to identify the DNA biomarker. Butland et al. mention that the use of a labeled DNA probe could be used to detect the biomarker(s), which would require some knowledge of the DNA sequence in the biomarker be known. In order to sequence each biomarker from a mixture of multiple biomarkers, each biomarker has to be amplified separately, which means multiple sets of primers with multiple sample runs. This is apparently an inefficient and an expensive means of detection.

Slater et al., U.S. Pat. No. 5,643,728, disclosed a marking method for a liquid comprising of a plurality of particles, which were identified by at least two signal means. One of the signal means was non-nucleic acid and the other was nucleic acid based. The nucleic acid marker was comprised of a plurality of single-stranded DNA oligonucleotides having sequences used as templates for PCR, and each such oligonucleotide comprised a variable region flanked by a first and second generic regions on either side of the variable region. In short, Slater et al. used multiple single stranded synthetic oligonucleotides or DNA templates as a marker and used one set of primers complementary to the generic flanking regions for PCR amplification. The amplified products were then sequenced to decipher the information contained within the marker. The method is excellent in the number of variations that can be obtained in the variable region of the oligonucleotides. For example, a 20 mer in the variable region can produce 4²⁰=1.09E12 variations. However, this method also has no tolerance for errors. A single base mistake in PCR amplification or sequencing can lead to a totally different conclusion.

In Sleat et al., GB 2390055, a methodology similar to Slater et al. is disclosed. A plurality of single stranded DNA having the same sequences were used as a security marker for cash transport boxes and explosive dye, and sequencing was used to decode hidden nucleic acid information. As in all other sequencing based decoding methodologies, a major flaw is in the accuracy of sequencing. It is well known that the first 15˜20 bases are not reliable using the widely used capillary electrophoresis (CE) based sequencing technology, which is a big concern for those DNA security markers with only 40˜60 bases long.

Although use of synthetic oligo DNA as security markers can generate enormous amount of variations as mentioned above, without an accurate detection/decoding of approximately a third of the content, the use of synthetic oligo DNA as security marker is great undermined by sequencing techniques.

The present invention discloses methods for the generation of a large quantity of unique DNA ID tags with ease and accurate detection methodology.

### SUMMARY

The present invention, discloses novel methods to produce a large number of security markers and the detection thereof.

One of the methods for producing a plurality of security markers comprises, providing a single double stranded DNA (dsDNA) template and a pool of rtDNA oligonucleotides complementary to the template, grouping primers in the pool of rtDNA oligonucleotides into a plurality of smaller subsets using combinatorial variation techniques; and generating a plurality of security markers from the plurality of smaller subsets of rtDNA oligonucleotides in the pool of rtDNA oligonucleotides, each of the smaller subsets defining a distinct security marker. The plurality of smaller subsets comprises at least two sequencably distinct rtDNA oligonucleotides. Generally, the DNA template is from about 50 bases to about 90,000,000,000 bases in length. Wherein the sequences of the pool of rtDNA oligonucleotides have lengths ranging from about 5 bp to about 100 bp in length.

In most embodiments of the methods the grouping of primers in said pool of rtDNA oligonucleotides into the plurality of smaller subsets of rtDNA oligonucleotides is carried out according to the equation;

n!/(Y!(n−Y)!).

wherein: n is the number of amplicons that can be generated by said pool of rtDNA oligonucleotides with a detection primer and the single DNA template; and Y is the number of amplicons generated by each of the plurality of smaller subsets of rtDNA oligonucleotides with a detection primer and the single DNA template.

In certain embodiments, the DNA template is selected from the group consisting of artificially synthesized oligo DNA, biosynthesized DNA from living organisms, extracted DNA from living organism, or a PCR product.

In other embodiments the method of generating security markers comprises, providing a first DNA fragment as a template, providing a pool of oligonucleotides having corresponding sequences to the first DNA fragment template; and generating, by combinatorial variations, a plurality of security markers each comprising a different grouping of oligonucleotides from the pool of oligonucleotides. Wherein the pool of oligonucleotides comprises a plurality of non-interfering rtDNA oligonucleotides having sequences complementary to the first DNA fragment template. Furthermore, the sequences of the pool of oligonucleotides have lengths ranging from about 5 bp to about 100 bp in length. Wherein the DNA template can be of any length greater than the length sum of one of the rtDNA oligonucleotides and a detection primer, preferably from a size range of about 50 bases to about 90 billion, usually ranges between about 100 bases to about 10 kilo bases, more usually about 500 bases to about 6 kb, and preferably about 1 kb to about 3 kb in length.

In yet another embodiment, the method further comprises providing at least one fluorescent labeling dye for signal detection of at least one of the security markers.

The method may also comprise providing a second DNA fragment as a template, the second DNA fragment template having a pool of oligonucleotides with sequences corresponding to the second DNA fragment template.

In most embodiments, the method further comprises providing a detection primer, wherein the rtDNA oligonucleotides and the detection primer produce a plurality of different-sized amplicons during PCR amplification. Wherein the fluorescent dyes are terminal oligonucleotide labeling dyes.

In other embodiments, the combinatorial variations are generated using the equation

n!/(Y!(n−Y)!)

wherein: n is the number of oligonucleotides in the pool of oligonucleotides; and Y is the number of oligonucleotides in each grouping used to form an individual security marker. Wherein the number of said groupings ranges from 1 to n, where n is the number of oligonucleotides in the pool of oligonucleotides.

The invention also provides security markers in accordance with the invention. In one embodiment, a security marker comprises, a plurality of oligonucleotides, the oligonucleotides complementary to a DNA template; wherein the oligonucleotides are chosen by a combinatorial variation technique from a pool of oligonucleotides complementary to the DNA template. Wherein the pool of oligonucleotides are non-interfering rtDNA oligonucleotides to the DNA template.

In certain embodiments, the rtDNA oligonucleotides are labeled with a fluorescent dye.

In most embodiments the security marker is a covert marker for individual product identification.

Generally, the combinatorial variation technique utilized for producing the security markers comprises grouping the pool of oligonucleotides by the equation the

n!/(Y!(n−Y)!)

where n is the number of possible amplicons that can be generated during PCR by the pool of oligonucleotides and a detection primer(s), and Y is the number of oligonucleotides in each grouping within n.

In most embodiments the security marker is a covert marker for individual product identification.

In some embodiments, the detection primer is included in the security markers.

A method for authenticating an article, comprising, selecting a security marker specific for the article to be authenticated, said security marker comprising a plurality of oligonucleotides derived from a pool of rtDNA oligonucleotides, applying the security marker to the article, collecting a sample of the security marker from the article, analyzing the oligonucleotides in the security marker using one DNA template complementary to the oligonucleotides in the security marker using PCR techniques, generating an amplicon length profile corresponding to the oligonucleotides in the security marker, comparing the amplicon length profile to a security marker profile database and determining if the amplicon length profile generated corresponds to the designated security marker associated with the article.

The method of authenticating an article, wherein the generating of a DNA polymorphic fragment length profile utilizes capillary electrophoresis.

The method of authenticating an article, wherein the pool of rtDNA oligonucleotides ranges from about 5 to about 200 unique rtDNA oligonucleotides.

The method of authenticating an article, wherein the oligonucleotides are selected from the pool of rtDNA oligonucleotides using combinatorial variation techniques.

All patents and publications identified herein are incorporated herein by reference in their entirety.

### BRIEF DESCRIPTION OF THE FIGURES

FIG. 1 is a flow chart showing one embodiment of the method of generating a large number of security markers using one PCR template, the produced security markers having a group of rtDNA oligonucleotides in accordance with the invention.

FIG. 2 is a flow chart showing one embodiment of the method of generating a large number of security markers using at least two PCR templates and two detection primers, the produced security markers having a group of rtDNA oligonucleotides in accordance with the invention.

FIG. 3 is a flow chart of one embodiment of the methods for authenticating an article utilizing a security marker of the invention.

FIG. 4 is a chart showing the 210 various combination of primers sets generated by the example shown in Table I in accordance with the methods of the invention.

FIG. 5 is a diagram showing the PCR amplicons generated from one embodiment of a security marker in accordance with the invention.

FIG. 6 is yet another diagram showing the polymorphic fragment lengths generated during PCR from a pool of rtDNA oligonucleotides in accordance with the invention.

FIGS. 7a and 7b are diagrams showing the PCR amplicons generated from one embodiment having two pools of primer sets and two templates in accordance with the invention.

FIG. 8 is a representation of electrogram of capillary electrophoresis showing a security marker consisting of five DNA combinations after being extracted from the ink of a security marker and PCR amplified in a single reaction in accordance with the invention.

### DESCRIPTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Optional” or “optionally” means that the subsequently described event or circumstance may but need not occur, and that the description includes instances where the event or circumstance occurs and instances in which it does not.

The terms “those defined above” and “those defined herein” when referring to a variable incorporates by reference the broad definition of the variable as well as preferred, more preferred and most preferred definitions, if any.

The term “primer” means a nucleotide with a specific nucleotide sequence, which is sufficiently complimentary to a particular sequence of a template DNA molecule, such that the primer specifically hybridizes to the template DNA molecule.

The term “probe” refers to a binding component which binds preferentially to one or more targets (e.g., antigenic epitopes, polynucleotide sequences, macromolecular receptors) with an affinity sufficient to permit discrimination of labeled probe bound to target from nonspecifically bound labeled probe (i.e., background).

The term “probe polynucleotide” means a polynucleotide that specifically hybridizes to a predetermined target polynucleotide.

The term “oligomer” refers to a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The term “PCR” refers to polymerase chain reaction. This refers to any technology where a nucleotide is amplified via a temperature cycling techniques in the presence of a nucleotide polymerase, preferably a DNA polymerase. This includes but is not limited to real-time PCR technology, reverse transcriptase-PCR, and standard PCR methods.

The term “nucleic acid” means a polymer composed of nucleotides, e.g. deoxyribonucleotides or ribonucleotides, or compounds produced synthetically which can hybridize with naturally occurring nucleic acids in a sequence specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide”, “oligo”,“polynucleotide” or “nucleotide” refer to single or double stranded polymer composed of nucleotide monomers of generally greater than 5 nucleotides in length.

The term “monomer” as used herein refers to a chemical entity that can be covalently linked to one or more other such entities to form an oligomer. Examples of “monomers” include nucleotides, amino acids, saccharides, peptides, and the like. The term nucleotide means

The term “identifiable sequence” or “detectable sequence” means a nucleotide sequence which can by detected by hybridization and/or PCR technology by a primer or probe designed for specific interaction with the template nucleotide sequence. The interaction of the template nucleotide sequence with the specific probe or primer can be detected by optical and/or visual means to determine the presence of the target nucleotide sequence.

The term “amplicon” means an oligonucleotide formed or produced during PCR amplification.

The term “polymorphic length fragments” mean nucleotide fragments which comprise some sequence homology with one another.

The term “reverse template DNA”, “rtDNA” means an oligonucleotide complementary to a DNA template. Generally, rtDNA is a primer, more specifically a “marking primer” which is included in a security marker in accordance with the invention.

The term detection primer means a primer utilized in PCR to form amplicons with the rtDNA (marking primer) in the security marker along with a DNA template in the PCR reaction. Generally, the detection primer is not in the security marker, but in some embodiments, the detection primer and the rtDNA oligonucleotides are included in the security marker.

The term “DNA template” means an oligonucleotide, synthetic or natural, which is used as a docking DNA fragment for primers with complimentary or corresponding sequences for PCR amplification. A DNA template can be single stranded or double stranded DNA.

The present invention discloses methods to generate a significant amount of DNA combinations for security markers from a single DNA template and the detection thereof. The present invention relates to methods for generating a large quantity of security markers from a small pool of primer sets and only one or a few DNA fragments used as PCR templates. By using primers in the security marker instead of the DNA template, large numbers of security markers can be generated by grouping the pool of primers into various subsets.

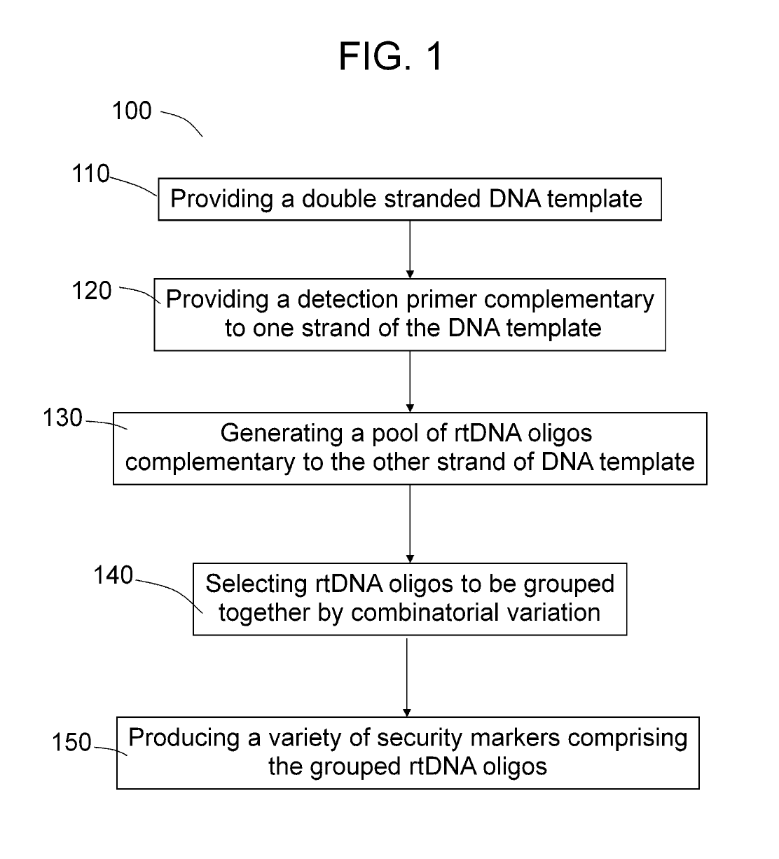

Referring to FIG. 1 is a flow chart of one embodiment of the methods of generating security markers in large quantities. The method 100 for generating security markers comprises providing a DNA template at event 110. The DNA template maybe a synthetic or natural occurring oligonucleotide fragment. The DNA template can be chosen from animals, plants, bacteria, viruses, fungi, or synthetic vectors or fragments or any combination thereof. The DNA template may have the size range of about 100 bases to about 90 billion, usually ranges between about 100 bases to about 10 kilo bases, more usually about 500 bases to about 6 kb, and preferably about 1 kb to about 3 kb in length.

In certain embodiments of the methods of the invention, the DNA template is derived from DNA extracted from a specific plant source and is specifically digested and ligated to generate artificial nucleic acid sequences which are unique to the world. The digestion and ligation of the extracted DNA is completed by standard restriction digestion and ligation techniques known to those skilled in the art of molecular biology.

The DNA template may also be single stranded (ssDNA) or double stranded (dsDNA) depending on which is preferred for the amplification technique to be utilized for analysis of the DNA in the security marker. The DNA template is not present in the security markers but is utilized to design and produce a plurality of corresponding oligonucleotides complementary to the DNA template as well as being utilized in PCR amplification.

The method 100 further comprises providing a detection primer at event 120. In this embodiment there is only one detection primer provided which corresponds to the DNA template. The length of the detection primer is from the range of about 5 bases to about 50 bases, more preferably about 15 to about 30. In most embodiments the detection primer comprises a fluorescent label to allow for the detection of amplicons produced during PCR. The fluorescent labels include but are not limited to Fam, Ned, Ted, and Rox.

The detection primer and the DNA template are not part of the security marker illustrated in FIG. 1, but are utilized for designing and generating a pool of reverse template DNA (rtDNA) oligonucleotides/marking primers complementary to the DNA template in event 130. The pool of rtDNA oligonucleotides are designed to avoid amplification problems such as primer dimmers, hairpin turns, or any other unwanted interference with the other rtDNA oligonucleotides in the rtDNA pool as well as with the detection primer during PCR. Detection primer and rtDNA are designed and simulated by electronic PCR program (Amplify 1.2 for example). All of the rtDNA oligonucleotides have unique DNA sequences complementary to the DNA template, allowing the formation of amplicons with unique lengths when using the detection primer during amplification. While in most embodiments the pool of rtDNA oligonucleotides have distinct oligonucleotide sequences, some of the rtDNA oligonucleotides can have overlapping sequences with one another as long as their total sequence is not identical and they are capable of producing unique amplicon lengths with the selected detection primer.

In some embodiments, the detection primer is a forward primer and the rtDNA oligonucleotides in the security marker(s) are reverse primers. In other embodiments the reverse occurs and the detection primer is a reverse primer and the rtDNA oligonucleotides or marking primers in the security marker are forward primers.

At event 140, method 100 comprises selecting or grouping the rtDNA oligonucleotides together by combinatorial variation. The number of possible combinatorial variations of grouped rtDNA oligonucleotides is determined by

n!/Y!(n−Y)!

where n is the number of unique amplicons or polymorphic length DNA fragments that can be produced from the detection primer and the pool of rtDNA oligonucleotides assuming one DNA template for PCR amplification; and Y is the number of marking primers in each particular group or subset of rtDNA oligonucleotides to be utilized in an individual security marker.

For example, with one detection primer and a pool of twenty rtDNA oligonucleotides, twenty unique primer sets (detection primer and a rtDNA oligonucleotide) are made which generate twenty corresponding amplicons with unique lengths which is “n” in the above equation. Generally, the pool of rtDNA oligonucleotides could be grouped from about 3 to about 10 rtDNA oligonucleotides per grouping; since the maximum number of combination is achieved when Y=n/2 for even number n and Y=(n−1)/2 or (n+1)/2 for odd number n, thus, Y is usually approximately n/2 for even n and approximately (n−1)/2 or (n+1)/2 for an odd number n. When the pool of twenty rtDNA oligonucleotides are grouped in three's the total combinatorial variation is 1,140 combinations. When the pool of twenty rtDNA oligonucleotides are grouped by 10 the combinatorial variation is 184,756 combinations. Thus generating from 1000 to about 185,000 variations of security markers is possible by using twenty rtDNA oligonucleotides, one detection primer and one DNA template in accordance with the methods of the invention.

Once the rtDNA oligonucleotides have been designed to correspond to the DNA template and the number of combinatorial variations of rtDNA oligonucleotides has been determined, the method of FIG. 1 further comprises producing a plurality of security markers comprising the grouped rtDNA oligonucleotides at event 150. By analyzing the PCR amplicons associated with the rtDNA oligonucleotides in the security marker for their base pair length/size, a security profile for a particular security marker can be detected and identified. Each item to be labeled with a security marker would have a unique combination of rtDNA oligonucleotides which generate specific amplicons that can be analyzed and identified by capillary electrophoresis. Thus each labeled item has an amplicon profile associated with a particular security marker, in a sense like a bar code to determine its authenticity and/or origin of the item.

The security marker comprises a group of rtDNA oligonucleotides which provide a unique amplicon size profile when produced by PCR and analyzed by capillary electrophoreses. The concentration of the rtDNA oligonucleotides in the security marker range from about 0.0025 uM to about 2.5 uM depending on how much sample is needed for PCR analysis and detection of the amplicon profile associated with a specific security maker.

In some embodiments extraneous oligonucleotides are also present in the security marker compound mixture to be able to camouflage or “hide” the specific rtDNA oligonucleotides in the marker with extraneous and nonspecific nucleic acid oligomers/fragments, thus making it difficult for unauthorized individuals, such as forgers to identify the sequence(s) of the rtDNA oligonucleotides in the security marker. In certain embodiments, the marker comprises genomic DNA from the corresponding or similar DNA source that was utilized to derive the rtDNA oligonucleotides. Such extraneous oligonucleotides may include but are not limited to virus, bacteria, yeast, fungus, plant, and animal.

In other embodiments, the security marker may also comprises the detection primer along with the designated group of rtDNA oligonucleotides. In this embodiment, only the DNA template appropriate buffers, enzymes and PCR solutions are needed to produce the amplicon profile associated with the group of rtDNA oligonucleotides in the security marker. When the detection primer is included in the security markers, in some embodiments it maybe fluorescently labeled and in other embodiments it is not. The rtDNA oligonucleotides within the security markers are preferably not labeled but like the detection primer, in certain embodiments are fluorescently labeled.

One example of a security marker in accordance with the invention is that the rtDNA oligonucleotides included in the security markers are reverse or 3′ primers and the detection marker used only for PCR is a forward 5′ primer. It should be noted that certain embodiments the reverse is possible and the rtDNA oligonucleotides in the security markers are forward primers or 5′ primers and the detection primer used for PCR is a reverse or 3′ primer.

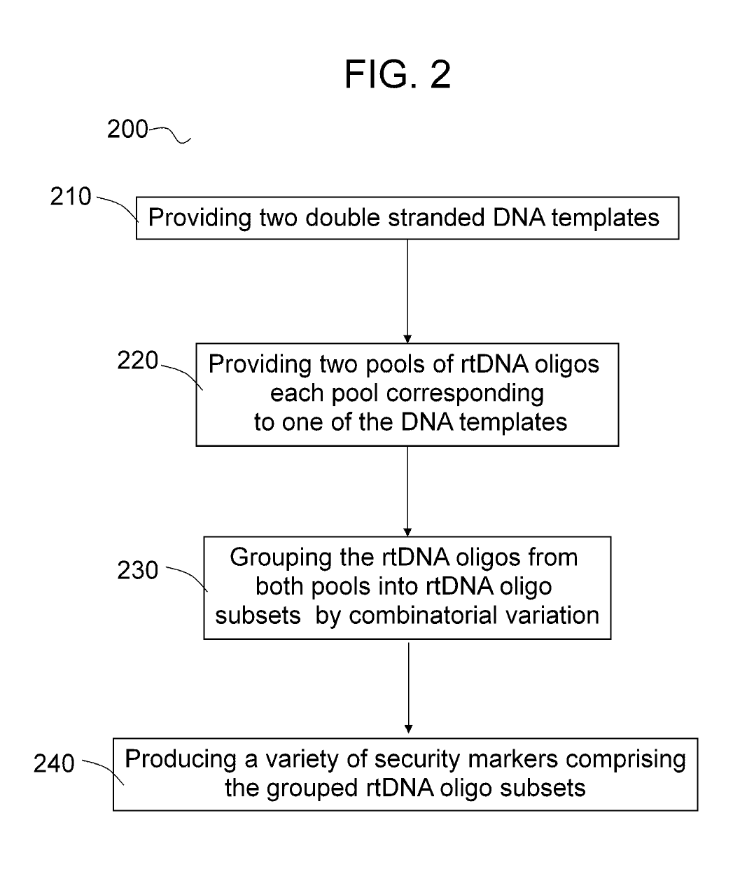

Referring to FIG. 2 is a flow chart showing an embodiment of the methods for generating large quantities of security markers. The method 200 comprises providing two DNA templates at event 210. The two templates maybe a synthetic or natural occurring oligonucleotide fragment. The DNA template can be chosen from animals, plants, bacteria, viruses, fungi, or synthetic vectors or fragments or any combination thereof. The DNA template may have the size range of about 50 bases to about 90 billion, usually ranges between about 100 bases to about 10 kilo bases, more usually about 500 bases to about 6 kb, and preferably about 1 kb to about 3 kb in length. Each template has unique and distinct flanking sequences for docking a plurality of primers.

At event 220, the method shown in FIG. 2 provides a pool of rtDNA oligonucleotides complementary to one of the two templates, a first DNA template (T1) and a second DNA template (T2). In this embodiment, there are two detection primers (F1 and F2), one corresponding to each template, respectively. The pool of marking primers or rtDNA oligonucleotides have two sub-pools of rtDNA oligonucleotides specific for an individual template. For example, if there were 30 rtDNA oligonucleotides (R1-R30) in the pool, 15 rtDNA oligonucleotides (R1-R15) would be unique for docking on the first DNA template (T1) and the other 15 rtDNA oligonucleotides (R16-R30) would be specific to the second DNA template (T2). Each of the amplicons produced during PCR by the rtDNA oligonucleotides sets, corresponding templates and detection primer in this embodiment have a unique length and can be detected and identified by capillary electrophoresis.

The various rtDNA oligonucleotides or rtDNA sets are grouped by combinatorial variation in event 230. Here the grouping of the rtDNA oligonucleotides are independent of which template they are complementary to. Using the following equation

n!/Y!(n−Y)!

where n is the total number of amplicons produced by the detection primer and rtDNA sets, e.g. F1 and R1-R15 as one sub-pool plus F2 and R16-30 as a second sub-pool, is “n” and Y is the number of rtDNA oligonucleotides sets grouped together to make a security marker in event 240. In the above embodiment, there could be, for example, thirty amplicons produced by two detection primers, two templates and 30 marking primers during PCR amplification. If there are 10 rtDNA oligonucleotides grouped together by combinatorial variation, over 30 million (3.0×10⁷) security markers are generated. That is, 30!/(10×(30−10)!).

At event 240 the security markers will not include the template but comprise a subset of rtDNA oligonucleotides and possibly the detection primers in certain embodiments. The rtDNA oligonucleotides in the security markers can be selected from either sub-pool or a combination thereof. The rtDNA oligonucleotides in the security marker are amplified by PCR with both templates and both detection primers present, thus allowing any of the specified rtDNA oligonucleotides to produce their corresponding amplicon(s) during PCR amplification.

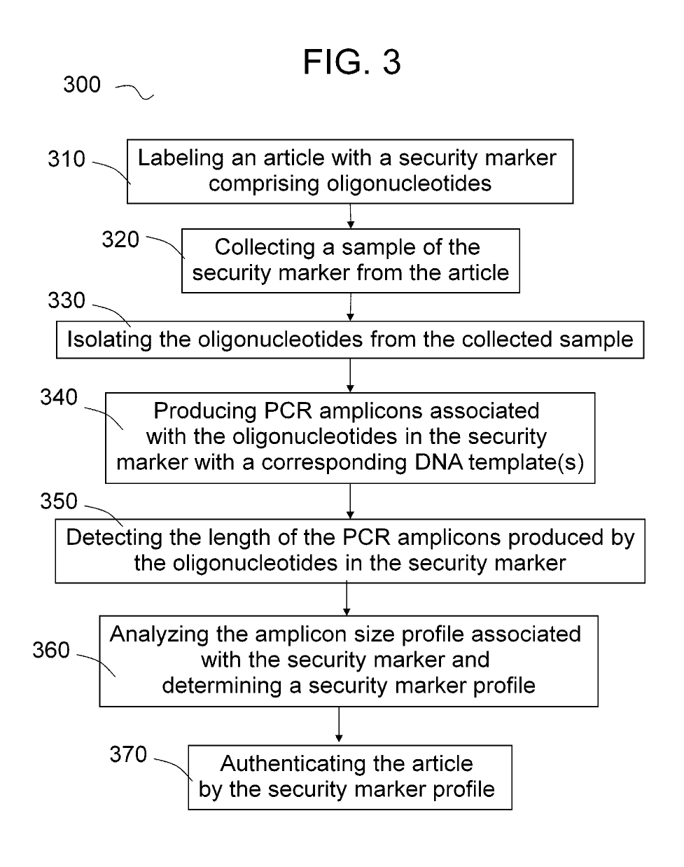

Referring to FIG. 3 is a flow chart showing one embodiment of the methods for authenticating an article with a security marker generated by combinatorial variation in accordance with the invention. The method 300 comprises labeling and article with a security marker comprising a plurality of oligonucleotides in event 310. The oligonucleotides are designed to hybridize to a specific DNA template in such a manner as to act as a primer to the template during PCR amplification. The oligonucleotides in the security marker have different sequences from one another allowing the production of various sized amplicons during PCR.

The method 300 further comprises 320 collecting a sample of the security marker from the article. Depending on the article, a portion of the security marker may be scrapped, chipped or dissolved away from the article.

In event 330 the oligonucleotides/primers are isolated or extracted from the collected sample to enable further analysis of the oligonucleotides. The collected sample maybe exposed to nucleic acid extraction buffer or similar solvents to isolate the DNA within the collected sample of the security marker.

The oligonucleotides isolated from the security marker on the article are amplified by PCR and the method 300 further comprises producing PCR amplicons associated with the oligonucleotides/primers using the specific DNA template in the PCR analysis in event 340. When the oligonucleotides in the security marker comprise only rtDNA oligonucleotides and not a detection primer, a detection primer is added to the PCR mixture along with the corresponding DNA template to enable amplicon production during PCR. Appropriate PCR buffers, dyes and or labels maybe added as needed to achieve PCR amplification with the primers collected from the security marker.

In other embodiments, the security marker comprises both the detection primer and a group of rtDNA oligonucleotides and thus the detection primer is already present in the collected sample and no additional detection primer is needed in the PCR mixture. In this embodiment, only the DNA template and appropriate PCR amplification mixtures are needed for amplification of the polymorphic length DNA fragments associated with the rtDNA oligonucleotides. It should be noted that the roles of the detection and marking primers can be interchanged, that is, the security marker may comprise a plurality of detection primers and then only one marking primer is utilized to produce the various sized amplicons during PCR.

In event 350, the method 300 further comprises detecting the PCR amplicons produced by the oligonucleotides in the security marker. In general, the amplicons generated by the oligonucleotides in the security marker are detected by capillary electrophoresis and analyzed by their length or size. Each security marker will generate a unique capillary electrophoresis profile depending on the oligonucleotides present in the security marker. The identification of the amplicon profile corresponds to a specific security marker for authentication.

In this invention, a single DNA template with a good selection of priming sites on both ends is selected. For each DNA template, the number of primers that can be designed is limited by the length of DNA fragment and sequence composition of the template, as long as the DNA sequences allows, in certain embodiments, more than 30 primer pairs can be generated along one DNA fragment.

For a single DNA template with 20 sets of primers, see Example 2, a combination of 4 out of 20 will generate 4,845 variations. By simply increasing the number of primer sets for same DNA template to 30 pairs, and keeping the grouping the same at 4 primers per group, the variation is calculated as 27,405. If the number or primers in each grouping or subset is increased from 4 to 6, when using a pool of 30 primers the number of variable combination results will be a combination of 593,775 for one template.

In other embodiments the number of templates are more than one, for example two templates can be analyzed each having 30 unique primer sets and grouping these primer set in groups of 6 will generate 50,063,860 variations, which shall be sufficient for most commercial applications.

The invention also provides kits for authenticating articles of interest using the methods of the invention. The kits of the invention may comprise, for example, a container of the nucleic acid extraction buffer, and a sample tube for holding a collected sample of the item or article to be authenticated. The kits may further comprise at least one detection primer and at least one DNA template configured to produce amplified PCR amplicons in the presence of corresponding rtDNA oligonucleotides extracted from a security marker. The kits may still further comprise a collection tool for taking a sample of the labeled article for transfer to the sample tube. The kits may further comprise a portable electrophoretic device (e.g. capillary electrophoresis system) for analyzing PCR products by length and/or size. The kits may further comprise an internal control for fragment size comparison for capillary analysis as well as a database of security marker profiles.

By way of example, the collection tool of the kit may comprise a blade or scissors for cutting a piece of the article, or the like. The sample tube of the kit may comprise a sealable vial or eppendorf tube, and may contain solvent or solution for extraction of the nucleic acids (e.g. DNA) from the sample taken from the article.

The kit may further comprise a DNA template, primer(s) and/or probes as well as solutions appropriate for PCR analysis. The kit may further comprise a small PCR instrument for analysis of the extracted nucleic acids from the article.

The capillary electrophoresis device of the kit may comprise an internal control for detecting the fragment size of the amplified PCR product(s).

In many embodiments, the kit will further comprise a system for accessing a data base of security marker amplicon profiles of interest, for comparison to the results obtained from the article. The kits of the invention thus provide a convenient, portable system for practicing the methods of the invention.

Preferred methods for generating security markers and authenticating articles utilizing the security markers are provided in the following Examples.

### EXAMPLES

The following preparations and examples are given to enable those skilled in the art to more clearly understand and to practice the present invention. They should not be considered as limiting the scope of the invention, but merely as being illustrative and representative thereof.

### Example I

Example 1 is a demonstration of the number of combinatorial variations of DNA markers generated from 1 DNA template, 10 primer sets with a group of 4 combinations.

As shown in Table I, one detection primer (F) and 10 rtDNA oligonucleotides (Rx) can generate 10 different DNA amplicons from one DNA template. Table I also shows the ten different primer sets generated by detection primer F and rtDNA oligonucleotides R-R10. These ten primer sets produced ten distinct polymorphic fragments which correspond to the DNA template.

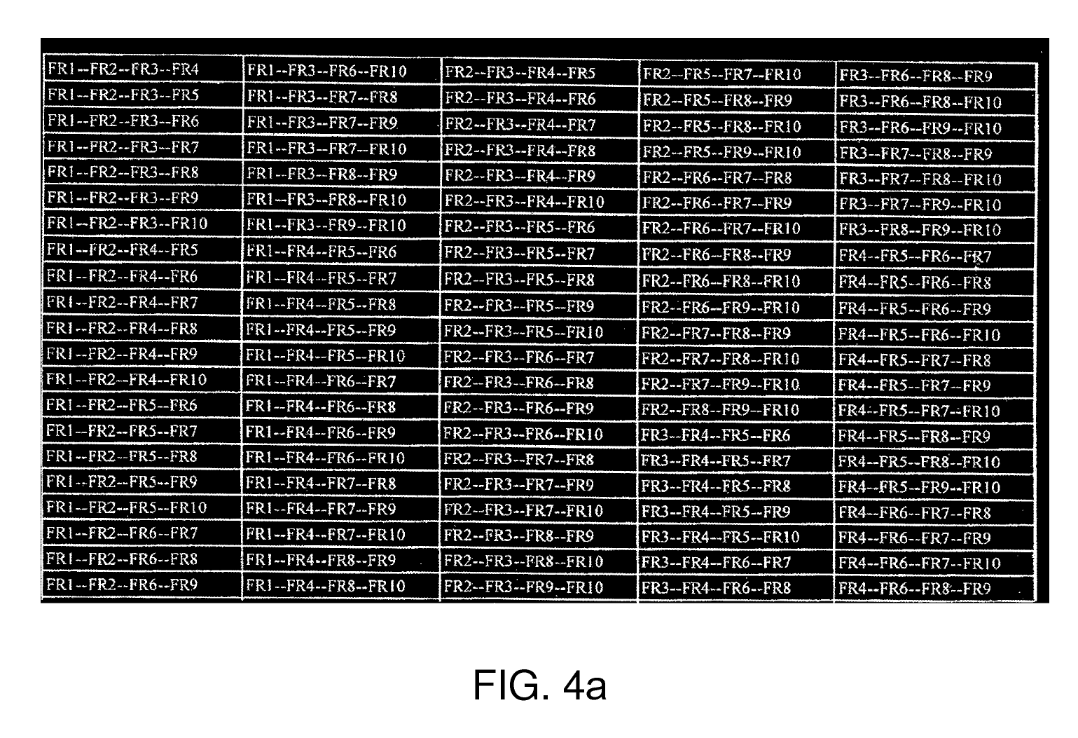

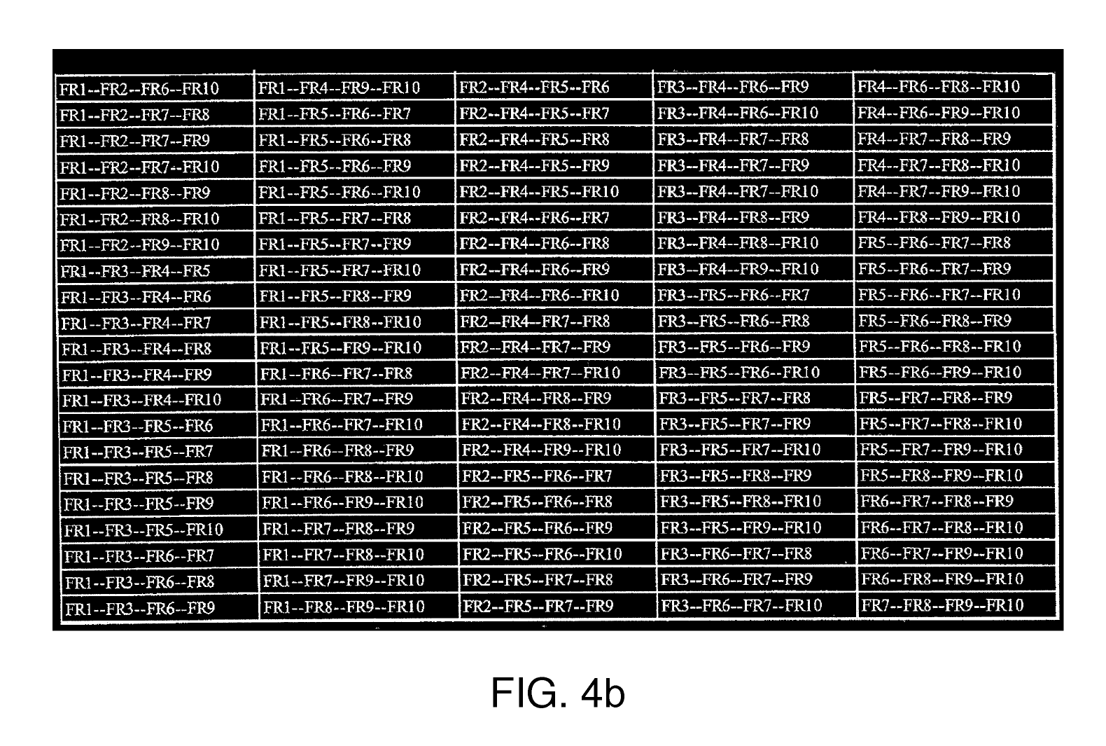

Therefore, the total variation of a 1×10 primers (1 detection×10 rtDNA) and a combination with a group or subset of 4 can produce a combinatorial variation of 210 (see FIG. 4).

TABLE I
Primer set variation of 1 detection and 10 marking primers

| | | | | | | | | | | |
|---|---|---|---|---|---|---|---|---|---|---|
| | R1 | R2 | R3 | R4 | R5 | R6 | R7 | R8 | R9 | R10 |
| F | FR1 | FR2 | FR3 | FR4 | FR5 | FR6 | FR7 | FR8 | FR9 | FR10 |

FIG. 4 provides a chart that is a graphical representation of the oligonucleotide/primer sets generated by combinatorial variation using 1 DNA template and 1 detection primer plus 10 rtDNA oligonucleotides from Table I. Each primer set contains the same detection primer and a different grouping of rtDNA oligonucleotides to achieve the 210 possible variations. These combinatorial variations can be used to form unique security markers based on the grouping of the rtDNA oligonucleotides alone or security markers also including the detection primer as well.

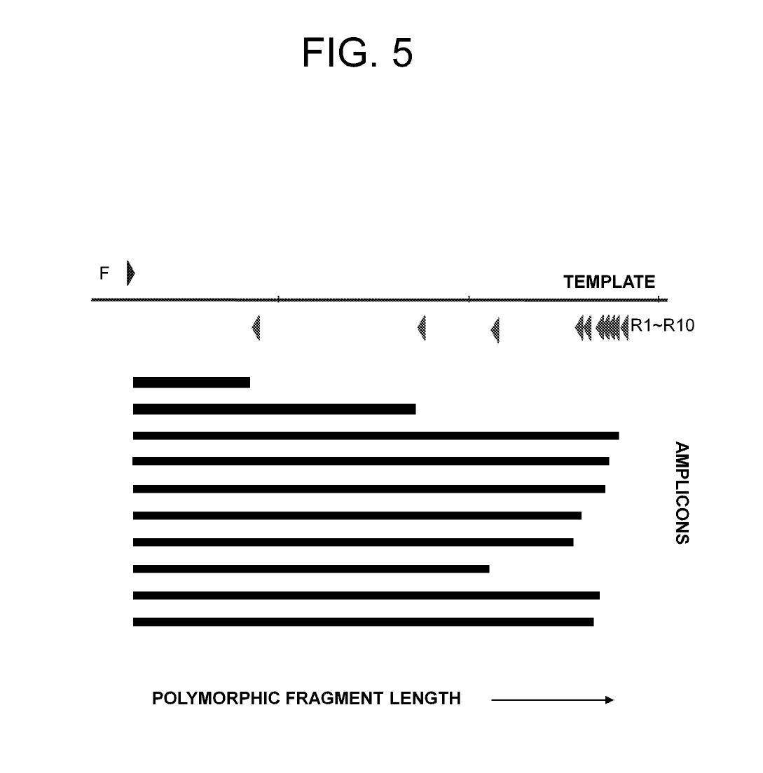

The oligonucleotides in the security markers are analyzed with PCR in the presence of the DNA template and the detection primer, if the rtDNA oligonucleotides is the only DNA present in the security marker. FIG. 5 shows a diagram of the polymorphic fragments generated by the oligonucleotide sets in TABLE 1 utilizing one template, one detection primer, and ten rtDNA oligonucleotides. A subset of oligonucleotides is produced by various combinations of the rtDNA oligonucleotides/marking primers shown in FIG. 5. Each subset of rtDNA oligonucleotides will make up a specific security marker that can then be analyzed by PCR. The PCR products produced by the rtDNA oligonucleotides in the security marker are then detected by capillary electrophoresis using capillary electrophoresis to determine the size of the amplicons corresponding to the rtDNA oligonucleotides in the security marker and identify the security marker amplicon profile.

### Example II

Example 2 demonstrates how many combinatorial variations of DNA security markers are generated from 1 DNA template, 20 oligonucleotide sets with a grouping of 5 rtDNA oligonucleotides per combination.

As shown in Table II, one detection primer (F) and 20 rtDNA oligonucleotides (R1˜R20) can generate 20 different sized DNA amplicons from one DNA template during PCR amplification. The total combinatorial variations that can be generated with a grouping of 5 rtDNA oligonucleotides is 20!/5!(20−5)!, which is 15,504 variations.

Table II shows the oligonucleotide set variations generated by 1 detection primer and 20 rtDNA oligonucleotides for one template to form the 20 oligonucleotide sets in this example. Over 15,000 security makers are generated using this embodiment of the methods of the invention when the 20 oligonucleotide sets are grouped in subsets of 5 rtDNA oligonucleotides and using one detection primer in PCR amplification.

TABLE II

| | | | | | | | | | | | |
|---|---|---|---|---|---|---|---|---|---|---|---|
| | R1 | R2 | R3 | R4 | R5 | R6 | R7 | R8 | R9 | R10 | R11 |
| F | FR1 | FR2 | FR3 | FR4 | FR5 | FR6 | FR7 | FR8 | FR9 | FR10 | FR11 |

| | | | | | | | | | |
|---|---|---|---|---|---|---|---|---|---|
| | R12 | R13 | R14 | R15 | R16 | R17 | R18 | R19 | R20 |
| F | FR12 | FR13 | FR14 | FR15 | FR16 | FR17 | FR18 | FR19 | FR20 |

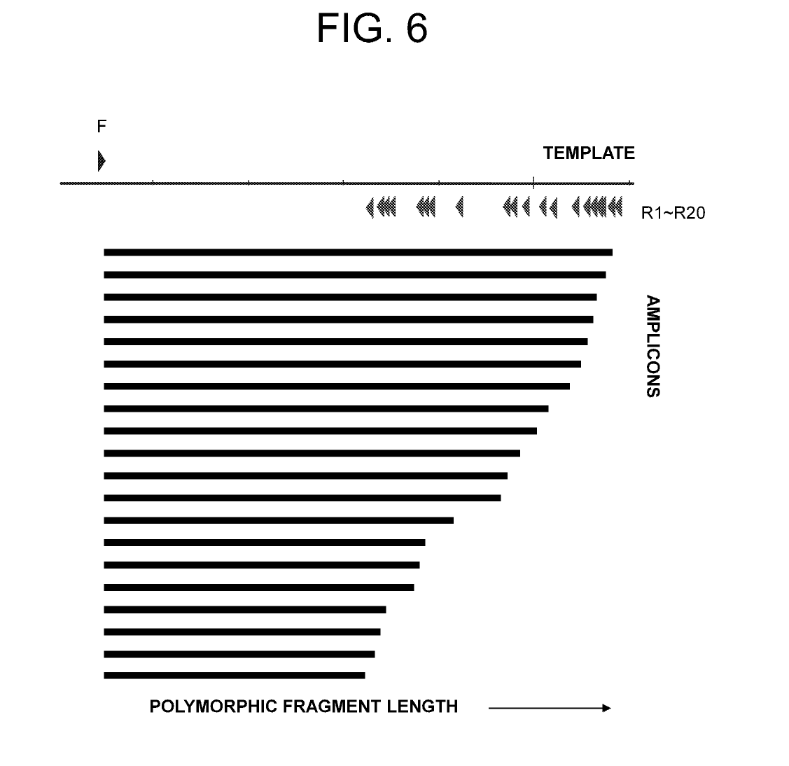

FIG. 6 shows a diagram of the polymorphic length fragments generated by the primers in TABLE 2 utilizing one template, one detection, and twenty rtDNA oligonucleotides. A subset of rtDNA oligonucleotides is produced by various combinations of the rtDNA oligonucleotides shown in FIG. 6. Each subset of rtDNA oligonucleotides will make up a specific security marker that is then be analyzed by PCR. The PCR products produced by the primers in the security marker are then detected by capillary electrophoresis using capillary electrophoresis to determine the size of the amplicons corresponding to the rtDNA oligonucleotides in the security marker and identify the security marker amplicon profile.

### Example III

Example 3 demonstrates the number of combinatorial variations of DNA markers generated from two DNA templates and 20 oligonucleotide sets with a combinatorial grouping of 5, and the detection thereof.

As shown in Table III, two detection primers (F1, F2) are utilized along with 20 rtDNA oligonucleotides (R1˜R20) to generate 20 different DNA amplicons from two DNA templates (T1, T2), and the total combinatorial variations of a 20 oligonucleotide sets with a group of 5 is 15,504 variations. In this example, each template has one corresponding detection primer and ten rtDNA oligonucleotides. Instead of having 20 rtDNA oligonucleotides set related to one DNA template as in Example 2, here 10 rtDNA oligonucleotides sets are utilized per template. This allows for larger complexity of the type of security makers that can be generated. Different templates and combination thereof can be designed for a specific customer and then stored in a security marker database for that specific customer. It is possible to “mix and match” the various templates and the corresponding rtDNA oligonucleotides for individual customers.

Table III shows the primer combination variation of 20 oligonucleotide sets for two templates T1 and T2.

TABLE III

| | | | | | | | | | | |
|---|---|---|---|---|---|---|---|---|---|---|
| T1 | R1 | R2 | R3 | R4 | R5 | R6 | R7 | R8 | R9 | R10 |
| F1 | F1R1 | F1R2 | F1R3 | F1R4 | F1R5 | F1R6 | F1R7 | F1R8 | F1R9 | F1R10 |
| T2 | R11 | R12 | R13 | R14 | R15 | R16 | R17 | R18 | R19 | R20 |
| F2 | F1R11 | F1R12 | F1R13 | F1R14 | F1R15 | F1R16 | F1R17 | F1R18 | F1R19 | F1R20 |

The template and primer sequences utilized in this example are given below.

Template sequence 1
(SEQ ID NO. 1)
ctaccgccttccttgtacaatcatctgatgatgtatcatctgactgcttt
tccactttcaccgtttacaaatagtttccaaataaaccccaacaaaaaag
agaaaggaaaaaataaataaaaaaaaagataaagatatcgttgggaatga
aattctgtcatttgtatcccctttgacagaaaacggagggatctattgat
gtattttaaaaattttatccgtcgggactgacggggctcgaacccgcagc
ttccgccttgacagggcggtgctctgaccaattgaactacaatcccaggg
aaataaagaaaagtgtacaacag
Template sequence 2
(SEQ ID NO. 2)
gaaccctctttactgttcaaagagaaaaaaaagtcttttttttatttaat
tttaataagatcttgccttagtgtagtcacatattagacacttaccccct
gttttatttgaatttcatttatgaaatgctttattgactcatttcatatc
atggatcaaagaagttaaattcaaaatcggcagggtatacccttttttcg
aaacgaaatacgaagaaaagttaccatagaactctttggattatatattc
ttttccaccctttcttttcgataatctaccgccttccttgtacaatcatc
tgat
Primer list
Detection primer 1, F1:
(SEQ ID NO. 3)
catctgatgatgtatcatctgactgc
Detection primer 2, F2:
(SEQ ID NO. 4)
gaaccctctttactgttcaaagag

| | | |
|---|---|---|
| R1: | GGATTGTAGTTCAATTGGTCAGAG | (SEQ ID NO. 5) |
| R2: | GTAGTTCAATTGGTCAGAGCACCG | (SEQ ID NO. 6) |
| R3: | TCAATTGGTCAGAGCACCGCCC | (SEQ ID NO. 7) |
| R4: | TGGTCAGAGCACCGCCCTGTCAAG | (SEQ ID NO. 8) |
| R5: | ACCGCCCTGTCAAGGCGGAAGCTG | (SEQ ID NO. 9) |
| R6: | CCTGTCAAGGCGGAAGCTGCGGGT | (SEQ ID NO. 10) |
| R7: | CAAGGCGGAAGCTGCGGGTTCGAG | (SEQ ID NO. 11) |
| R8: | CGGAAGCTGCGGGTTCGAGCCCC | (SEQ ID NO. 12) |
| R9: | CCCGTCAGTCCCGACGGATAA | (SEQ ID NO. 13) |
| R10: | TCAATAGATCCCTCCGTTTTCTG | (SEQ ID NO. 14) |
| R11: | AACAGGGGGTAAGTGTCTA | (SEQ ID NO. 15) |
| R12: | AAGGGTATACCCTGCCGATTTTG | (SEQ ID NO. 16) |
| R13: | GAGTTCTATGGTAACTTTTCTTCG | (SEQ ID NO. 17) |
| R14: | GGTAGATTATCGAAAAGAAAGGGTGG | (SEQ ID NO. 18) |
| R15: | GGCGGTAGATTATCGAAAAGAAAGG | (SEQ ID NO. 19) |
| R16: | AGGAAGGCGGTAGATTATCGAAA | (SEQ ID NO. 20) |
| R17: | GTACAAGGAAGGCGGTAGATTATCG | (SEQ ID NO. 21) |
| R18: | TGTACAAGGAAGGCGGTAGATTA | (SEQ ID NO. 22) |
| R19: | TGATTGTACAAGGAAGGCGGTAGAT | (SEQ ID NO. 23) |
| R20: | GATGATTGTACAAGGAAGGCGG | (SEQ ID NO. 24) |

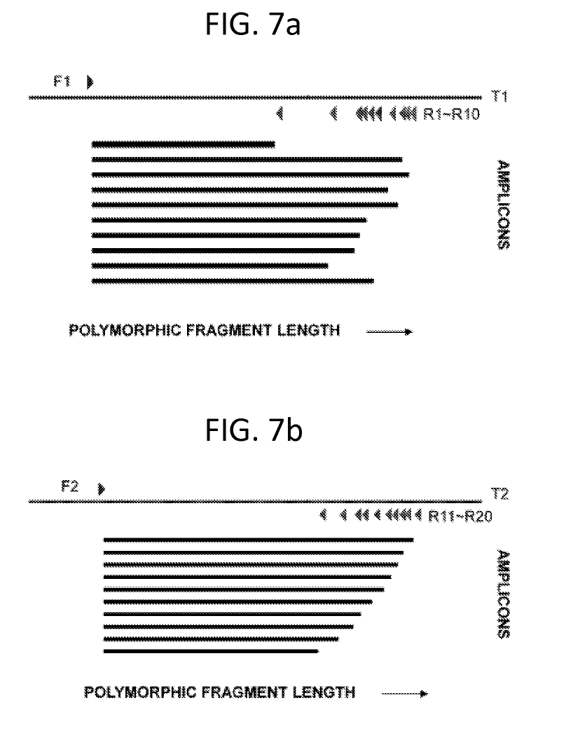

FIG. 7a is a graphical representation of the length polymorphic fragments (e.g. amplicons) generated by primers F1 and R1˜R10 with template 1. FIG. 7b is a similar graphical representation of the length polymorphic fragments generated by primers F2 and R11˜R20 with template 2. FIGS. 7a and b show the various lengths of the possible amplicons that can be generated during PCR in the presence of both templates and both detection primers. As long as the possible polymorphic fragments have at least 1 bp difference in length, they can be identified by capillary electrophoresis techniques.

In this example, the security marker comprises two detection primers and 5 rtDNA oligonucleotides out of the 20 rtDNA oligonucleotides available. The security marker is then added to a “cash-in-transit” ink for security applications. When the cash carrying box is tempered with, the ink will spray onto the cash to mark it with the security marker. When the cash is subsequently recovered a sample of the security marker is collected for identifying the cash by the amplicons produced by the rtDNA oligonucleotides in the security marker, thus linking the cash to a possible crime.

In general, cash samples are subjected to DNA extraction, which is commonly known to those skilled in forensic DNA sciences, and undergoes PCR amplification. Template (T1 and T2) sequences and detection primers are provided as an example for PCR amplification and the amplicons are analyzed by capillary electrophoresis with 5 dye settings. Amplicon sizes are analyzed and compared to the database for identification.

PCR thermocycle scheme; first cycle, 3′ for denaturing at 94° C., 30 cycles of 94° C. for 30″, 50° C. for 20″, and 72° C. for 30″, followed by 5 min at 72° C.

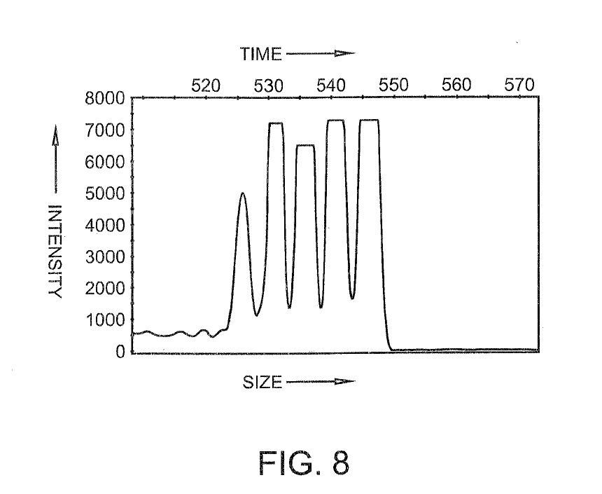

FIG. 8 is an electrogram of a security marker having five amplicons being detected by capillary electrophoresis after rtDNAs were extracted from the ink and PCR amplified in a single reaction. The five rtDNA oligonucleotides produce five amplicons having unique sizes which are easily detected by polymorphic fragment analysis. The electrogram shown in FIG. 8 shows the security marker profile for this particular security marker which can be stored in a database for future reference.

1. A method of producing a plurality of security markers, the method comprising:
providing a single DNA template;
providing a pool of rtDNA oligonucleotides complementary to the template;
grouping primers in said pool of rtDNA oligonucleotides into a plurality of smaller subsets using combinatorial variation techniques; and
generating a plurality of security markers from said plurality of smaller subsets of rtDNA oligonucleotides in the pool of rtDNA oligonucleotides, each of the smaller subsets defining a distinct security marker.

2. The method of claim 1, wherein each of the plurality of smaller subsets comprise at least two sequenceably distinct rtDNA oligonucleotides.

3. The method of claim 1, wherein the DNA template is from about 50 bases to about 90,000,000,000 bases in length.

4. The method of claim 1, wherein the grouping of primers in said pool of rtDNA oligonucleotides into the plurality of smaller subsets of rtDNA oligonucleotides is carried out according to the equation
n!/(Y!(n−Y)!)
wherein: n is the number of amplicons that can be generated by said pool of rtDNA oligonucleotides with a detection primer and the single DNA template; and Y is the number of amplicons generated by each of the plurality of smaller subsets of rtDNA oligonucleotides with a detection primer and the single DNA template.

5. The method of claim 1, wherein the DNA template is selected from the group consisting of an artificially synthesized DNA, a biosynthesized DNA from a living organism[s], extracted DNA from a living organism, or a PCR product.

6. A method of generating security markers comprising:
providing a first DNA fragment as a template,
providing a pool of oligonucleotides having corresponding sequences to the first DNA fragment template; and
generating, by combinatorial variations, a plurality of security markers each comprising a different grouping of oligonucleotides from the pool of oligonucleotides.

7. The method of claim 6, wherein the pool of oligonucleotides comprises a plurality of non-repeat rtDNA oligonucleotides having sequences complementary to the first DNA fragment template.

8. The method of claim 6, wherein the sequences of the pool of oligonucleotides have lengths ranging from about 5 bp to about 100 bp in length.

9. The method of claim 6, further comprising providing at least one fluorescent labeling dye for signal detection of at least one of the security markers.

10. The method of claim 6, further comprising providing a second DNA fragment as a template, the second DNA fragment template having a pool of oligonucleotides with sequences corresponding to the second DNA fragment template.

11. The method of claim 7, further comprising providing a detection primer, wherein the rtDNA oligonucleotides and the detection primer produce a plurality of different sized amplicons during PCR amplification.

12. The method of claim 9, wherein the fluorescent dyes are terminal oligonucleotide labeling dyes.

13. The method of claim 6, wherein the combinatorial variations are generated using the equation
n!/(Y!(n−Y)!)
wherein: n is the number of oligonucleotides in the pool of oligonucleotides; and Y is the number of oligonucleotides in each grouping used to form an individual security marker.

14. The method of claim 13, wherein the number of said groupings range from 1 to n, where n is the number of oligonucleotides in the pool of oligonucleotides.

15. A security marker comprising:
a plurality of oligonucleotides, said oligonucleotides complementary to a DNA template;
wherein said oligonucleotides are chosen by a combinatorial variation technique from a pool of oligonucleotides complementary to the DNA template.

16. The security marker of claim 15, wherein said pool of oligonucleotides are nonrepeat rtDNA oligonucleotides to said DNA template.

17. The security marker of claim 16, wherein the rtDNA oligonucleotides are labeled with a fluorescent dye.

18. The security marker of claim 15, wherein the security marker is a covert marker for individual product identification.

19. The security marker of claim 15, wherein the combinatorial variation technique comprises grouping the pool of oligonucleotides by the equation
n!/((Y!(n−Y)!)
where n is the number of possible amplicons that can be generated during PCR by the pool of oligonucleotides and a detection primer, and Y is the number of oligonucleotides in each grouping within n.

20. The security marker of claim 19, wherein the detection primer is fluorescently labeled.

21. The security marker of claim 19, wherein the detection primer is included in the security markers.

22. A method for authenticating an article, comprising:
selecting a security marker comprising oligonucleotides for the article to be authenticated, said oligonucleotides derived from a pool of rtDNA oligonucleotides;
applying said security marker to the article;
collecting a sample of the security marker from the article;
analyzing the oligonucleotides in the security marker using PCR techniques with one DNA template complementary to the oligonucleotides in the security marker and a detection primer;
generating an amplicon length profile corresponding to the oligonucleotides in the security marker;
comparing the amplicon length profile to a security marker profile database; and
determining if the amplicon length profile generated corresponds to the security marker associated with the article.

23. The method of claim 22, wherein the generating an amplicon length profile utilizes capillary electrophoresis.

24. The method of claim 22, wherein the pool of rtDNA oligonucleotides ranges from about 5 to about 100 unique rtDNA oligonucleotides.

25. The method of claim 22, the oligonucleotides are selected from the pool of rtDNA oligonucleotides using combinatorial variation techniques.

26. The method of claim 1, wherein the sequences of the pool of rtDNA oligonucleotides have lengths ranging from about 5 bp to about 100 bp in length.

27. The method of claim 6, wherein the DNA template can be of any length greater than the length sum of one of the rtDNA oligonucleotides and a detection primer, preferably from about 50 bases to about 90,000,000,000 base pairs.
